(19) United States
(12) Patent Application Publication
(10) Pub. No.: US 2008/0058442 A1
(43) Pub. Date: Mar. 6, 2008

(54) Two-Step System For Improved Initial And Final Characteristics Of A Biomaterial

(76) Inventors: Leif Hermansson, Molle (SE); Hakan Engqvist, Knivsta (SE); Hakan Spengler, Uppsala (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/577,277

(22) PCT Filed: Oct. 29, 2004
(86) PCT No.: PCT/SE04/01577

§ 371 Date: May 14, 2007

(30) Foreign Application Priority Data
Oct. 29, 2003 (SE) 0302844-6
Apr. 22, 2004 (SE) 0401026-0

(51) Int. Cl. A61K 6/06

(52) U.S. Cl. 523/115; 106/35; 523/116

(57) ABSTRACT

A two-step system for chemically bonded ceramic (CBC) materials, and especially a dental filling material or an implant material. The system includes an initial working part-system to provide for improved early-age properties and a second main system to provide for improved end-product properties including bioactivity. The systems interact chemically. The invention also relates to the powdered materials and the hydration liquid, respectively, as well as the formed ceramic material.

### TECHNICAL FIELD OF THE INVENTION

The present invention relates to a system for chemically bonded ceramic (CBC) materials, preferably a dental filling material or an implant material, comprising a two-step procedure. This system includes an initial working part-system to provide for improved early-age properties and a second main system to provide for improved end-product properties including bioactivity. The systems interact chemically. The invention also relates to the powdered materials and the hydration liquid, respectively, as well as the formed ceramic material.

### BACKGROUND ART

The present invention relates to binding agent systems of the hydrating cement system type, in particular cement-based systems that comprise chemically bonded ceramics in the group that consists of aluminates, silicates, phosphates, carbonates, sulphates and combinations thereof, having calcium as the major cat-ion, and in addition to said system a second early age binding system is included. The invention has been especially developed for biomaterials for dental and orthopaedic applications, both fillers and cements as well as implants including coatings and carriers for drug delivery, but can also be used as fillers in industrial applications in electronics, micro-mechanics etc or in the construction field.

For materials, such as dental fling materials and implants, that are to interact with the human body, it is an advantage that the materials are made as bioactive or biocompatible as possible. Other properties that are required for dental filling materials and implants are a good handling ability with simple applicability in a cavity, moulding that permits good shaping ability, hardening/solidification that is sufficiently rapid for filling work without detrimental heat generation and provides serviceability directly following therapy, high hardness and strength, corrosion resistance, good bonding between filling material and biological wall, dimensional stability, radio-opacity, good long time properties and good aesthetics especially regarding dental filling materials. For the purpose of providing a material that fulfils at least most of these required properties, materials have been developed, such as those described in e.g. SE 463,493; SE 502,987; WO 00/21489; WO 01/76534; WO 01/76535; PCT/SE02/01480; and PCT/SE02/01481.

### SUMMARY OF INVENTION

This invention relates especially to the combination of improved early-age properties (properties achieved within the first ten minutes up to some hours) and the property development towards the final stage, which for different properties are achieved after some days or weeks. The present invention specifically relates to the problems of initial moulding ability, initial strength, heat evolved and early colour/transmittance development as well as high strength, viscoelasticity and other mechanical properties, i.e. the problem of enabling optimisation of a complex property profile in a bioactive product, and at the same time, also of the property profile of a the system during processing of the same to form the product.

The chemically bonded ceramic system for dentistry based on calcium aluminate minerals has two drawbacks related to initial strength and possible expansion. The final strength is reached after about 7 days, but the strength during the first hour is lower than that of a temporary filing material. The magnitude of the expansion may be too high not to raise questions from the dental community. According to ISO 1559 an amalgam restorative should have a dimensional stability within—0.15 to +0.2 linear %. The level 0.2% can be obtained in the Ca-aluminate based system, but expansion close to zero is desirable.

For orthopaedic applications an additional question deals with the heat evolved during the initial setting and hardening. This is more pronounced for treatments where larger amounts of biomaterial are injected.

The present invention addresses these issues for biomaterials based on chemically bonded ceramics. A low initial strength can cause failures during the first 24 hours and a somewhat too high expansion may cause tooth cracking in weakened teeth after the replacements of earlier fillings. The crucial question is how to increase the initial strength without affecting the final properties negatively, and is not a straightforward matter and demands a careful microstructural design. The use of two periods with different chemistry involved as in the present invention solves the problem with initial desired features of the biomaterial and the end-product characteristics.

Accordingly, the present invention aims at providing a system for CBC-based materials, preferably biomaterials, having improved controllability concerning its initial viscosity and consistency as well as heat evolved upon mixing of the powdered material and the hydration liquid of the system and early-age properties (initial strength, pore closure, translucency and early obtained bioactivity) and optimal end-product properties such as mechanical properties including compressive and bending strength and a sufficiently high E-modulus, a certain viscoelasticity and appropriate hardness, in the hydrated CBC-based product. This combination of improved initial properties and final properties is achieved by using an optimised combination of chemically compatible systems, where the first system is working in the initial phase in combination with the main system. The overall system works with pH-changes that are set by the selected part systems. The present invention is related to a pH controlled combination of a rapidly formed phase, primarily controlled by cross-linking chemistry and an overall acid-base reaction of chemically bonded ceramic type, primarily controlled by hydration chemistry. The control of pH is essential in transforming the initial acid system into a bioactive system, i.e. conditions for apatite formation. The rapid change into high pH-values reduces the risk of metal release.

These and other objectives are attained by the system, the powdered material (i.e. the inorganic binding phase and reactive glass), the hydration liquid and the ceramic material according to the invention, as defined in the claims.

According to one aspect of the invention, the powdered material and/or the hydration liquid comprises an additive of polyacrylic acid and/or a salt thereof or other polycarboxylic acids, co-polymers thereof, or polycarboxylates (i.e. a salt or ester of a polycarboxylic acid), all of which refer to the PAA-system.

By the inventive addition of a polycarboxylic acid or a copolymer or a salt or an ester thereof in the powdered material and/or in the hydration liquid, the following reactions take place during dissolving, hydration and polymerisation, here exemplified by a reaction between poly(acrylic-co-maleic acid) and calcium aluminate. R can be any group one ion (i.e. H⁺, Li⁺, Na⁺, K⁺, Rb⁺, preferably H⁺, Na⁺and K⁺) or NH₄⁺, and M could be a metal ion (e.g. Al³⁺, Ca²⁺, Sr²⁺, Si⁴⁺).

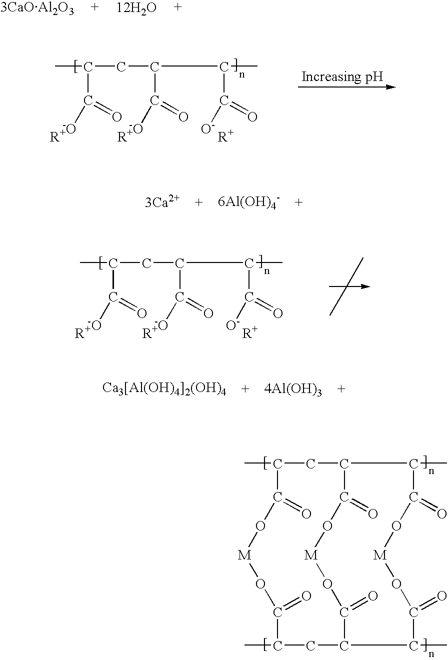

The organic hydrophilic system is not restricted to PAA-systems, but may also be based on other polycarboxylic acids, e.g. poly(maleic acid), poly(itaconic acid) or tricarballylic acid) or carboxylates such as phosphate esters. Also, polymers such as PAA/PEG can be used.

The source of the cross-linking metal ions (Ca, Al, Si, Sr . . . ) is addition of reactive glasses and the Ca-based cement material. Reactive glasses are preferably water soluble silicate glasses with Ca, Sr and/or Al as substitute ions for Si, e.g. glasses of the basic system (CaO SrO,Al₂O₃)-SiO₂ with high divalent ions contents.

The function of the poly acrylic acid or a salt (PAA) thereof can be divided into dispersing ability and cross-linking. As is understood, in the case with the cross-linking poly acid, the powdered material (the reactive glass and the calcium based cement material) is first dissolved in the liquid, thereafter Ca- and Al-ions cross-links the polyacrylic acid to form a polyacrylate polymer, and other Ca- and Al-ions hydrate to form hydrated calcium aluminate material in a second step. The resulting, hydrated material is a composite of CBC material and a cross-linked polyacrylate polymer. For an optimised formation of the two part composite—a biomer—the CBC system requires Ca-aluminate or Ca-silicate, reactive glass, e.g. of glass ionomer type, the composition of which is at least as soluble as traditional bioactive glasses, a poly acrylic acid and/or a salt thereof and inert filler particles, e.g. dental glass. The initial low pH of the system induces a dissolution of both the reactive glasses and the basic Ca-aluminate system or other chemically bonded ceramics of the same type, e.g. Ca-silicates.

Thus, binding phases may work during separate periods of time, or overlapping periods of time in the overall hardening process facilitating the combination of potential early-age properties with high performance end features especially related to biomechanical and biochemical properties.

### DETAILED DESCRIPTION OF THE INVENTION

As compared to the survey article on medical and scientific products by L. H. Hench “Engineered Materials Handbook” Vol 4, ASM International 1991, pp1007-1013, (especially Figures 1 and 2, p. 1008), the present invention deals with bioactive materials of an additional type, the type of which could be defined as type 5, i.e. with even faster dissolution and precipitation of phases than in the traditional bioactive glasses and/or resorbable materials. This is accomplished by the use of soluble glasses and the inorganic cement.

One route according to the present invention that yields surprisingly good initial results and improved final properties is to make a hybrid material of a glass ionomer cement and minerals of calcium aluminate and/or calcium silicate, maintaining a bioactive feature of the system. Glass ionomer cements consist of glass and poly acrylic acid. The acid dissolves the glass, and the ions from the glass cross-link the acid, and the material hardens. The reaction is rather rapid and nearly final strength is reached after about one hour. By exchanging fractions of the glass for calcium aluminate or silicate and a corresponding fraction of the PAA for water (with accelerator) a hybrid material can be formed. The liquid contents are controlled via

$$\frac{{w}_{c}}{c} + \frac{P A A}{reactive\_glass} + \frac{{w}_{G I C}}{reactive\_glass}$$

with a 0.2<w_{c}/c<0.45 (refers to the inorganic cement system), 0<PAA/(reactive glass)<0.21 and 0.2<w_{GIC}/(reactive glass)<0.45 (refers to the glass ionomer system). All ratios refer ratios by weight.

In the formula c=inorganic cement;

w_{c}=water to react with inorganic cement;
w_{GIC}=water to react with reactive glass, and
w (i.e. total water)=w_{c}+w_{GIC}.

The PAA can be applied as a solution and/ or as solid acid component.

Since the initial pH is acidic, the PAA reaction occurs first and as the acid is cross-linked the pH increases and the hydration of the Ca-aluminates continues. The material has a much higher initial strength than that of the pure ceramic system. The final strength is higher than that of the GIC. The microstructural variables are controlled by the reactive glass, the poly acrylic acid including the pH, the Ca-aluminate or Ca-silicate and inert fillers, e.g. dental glass particles or glass fibers.

The initial solution should have a pH<7, preferably 1-4, enhancing the cross-linking of the polycarboxylic. The pH increases when the polycarboxylic system meets the CA-system, resulting in a basic overall system at pH>7. The amounts of the polyacrylic acids are controlled to maintain pH<7 up to 30 minutes. After final hydration the pH approaches neutrality from the basic side. One problem with pure Glass Ionomer systems, which are based on polycarboxylic is the corrosion resistance sensitivity. The basic CAH system neutralises the initial acidity in the polyacrylic systems. The present invention could be looked upon as a two-phase biomaterial composed of two different biomaterials where the first is activated to take care of necessary early-age phenomena and the second biomaterial to establish the property profile of the end-product, included being a bioactive material.

The control of pH, especially the effect of obtaining a pH>7 early in the process—after initial acidic condition—is essential in transforming the initial acid system into a bioactive system, i.e. conditions for apatite formation, the requirements of which is high pH and a chemical surrounding of ions including calcium, phosphate and hydroxyl ions—the phosphate ions originating from phosphate glass, body liquid or from P-containing bonding materials, the hydroxyl ions from the dissolution of the Ca-aluminate system or added bases, preferably Li-hydroxide and/or Ca-hydroxide. The high pH contributes to formation of aluminate ions (Al(OH)₄-) instead of aluminium ions (Al³⁺).

Reactive filler particles in the present invention are composed of reactive glass, a phosphorous-containing glass and chemically bonded ceramics, preferably Ca-aluminates, preferably CA=(CaO)(Al₂O₃), C₁₂A₇=(CaO)₁₂(Al₂O₃)₇) and C₃A=(CaO)₃(Al₂O₃) and/or CS=(CaO SiO₂), C₂S=(2CaO SiO₂), and C₃S=(3CaO SiO₂), the latter preferably for orthopaedic applications. The composition of the reactive glass, especially the dissolution rate, is crucial. The glass grain size is also important and should be below 40 micron. The pure PAA gives an earlier general cross-linking reaction. Addition of a salt of the PAA is important in achieving improved viscosity at a low w/c. The inert filler is essential for the general end-product microstructure. Its effect concerns a lowered expansion, increased radio-opacity and favoured mechanical properties, especially hardness and fracture toughness.

Concerning calcium aluminate phases it is preferable to use CA, C₁₂A₇ and C₃A, which yield good initial strength. The addition of accelerator is dependant upon the selection of the Ca-aluminate phase. Low concentrations of lithium ions increase the reaction rate for CA. For C₁₂A₇ and C₃A the effect of accelerator is more complex.

According to another aspect of the invention addition of a base is included to achieve a change of pH to a high pH>7, more preferably pH>10 after an initial “acidic” time period of approximately 5 minutes. This is to assure an optimised hydration speed.

According to another aspect of the invention addition of a further acid is included to keep the pH<7 during a prolonged time of up to 30 minutes. This is to assure an optimised time for complete cross-linking of the acid.

Ways to induce such additional (delayed and then rapid) pH changes include release of acids/bases from a porous material (preferably nano/meso-pore structure or zeolite type structures). An additional way is coating of the particle surfaces to control the release/dissolution of pH changing species, especially the CBCs material, e.g. Ca-aluminate phases by coating with for instance Na-glyconate.

The active acids can be introduced either as dried substance together with the inorganic cement or as liquid in the hydration liquid or as a combination of both dry an active acid raw material and a liquid solution of the active acid.

Suitably, said polycarboxylic has a molecular weight of 100-250,000, preferably 1000-100,000 and it is present in an amount of up to 30%, preferably 1-20% and most preferred 3-15% by weight, calculated on the powdered material including any dry additives for dental applications.

It is preferred that the system comprises inert dental glass, as an additive in the powdered material, preferably at a content of 3-30 weight-% more preferred 5-20%. The particle size is critical in establishing high homogeneity. It is preferred that the particle size is 0.1-5 μm, more preferable 0.2-2 μm, and most preferable 0.3-0.7 μm. The dental glass may contain low additional amounts of less stable glass or reactive glass, preferable below 10% of the glass content. These glasses can preferably contain fluorine and phosphorus to yield fluoride ions, which contribute to F-apatite formation. According to the present invention the translucency is achieved earlier than in a pure an inorganic cement based system due to early pore closure.

Said polyacrylic acid or salt thereof is an acid in the group that consists of PAA, Me(I)-PAA, PAMA and Me(I)-PAMA, wherein

### PAA=poly acrylic acid

### PAMA=poly(acrylic-co-maleic acid)

### Me(I)-PAMA=poly(acrylic-co-maleic acid) Me(I)-salt

### Me(I)-PAA=poly acrylic acid Me(I)-salt

Me(I)=alkali metal ion, e.g. Na, K or Li

In one embodiment of the invention, at least a part or most preferred all of the reactive groups in the polycarboxylic based material bond to the CBC system.

The system may comprise one or more expansion compensating additives adapted to give the ceramic material dimensionally stable long-term attributes, as is described in WO 00/21489. Other additives and aspects of the system may follow that which is described in SE 463,493, SE 502,987, WO 00/21489, WO 01/76534, WO 01/76535, PCT/SE02/01480 and PCT/SE02/01481, the contents of which are incorporated herein by reference. For example, it is preferred at least for dental filling materials that the system comprises additives and/or is based on raw materials that contribute to translucency of the hydrated material.

According to one aspect of the invention the inert filler particles are composed of pre-hydrated chemically bonded ceramics of the same composition as the main binding phase. This improves the homogeneity of the microstructure and enhances the binding between reacting chemically bonded ceramics and the filler material.

According to another aspect of the present invention an additional system can be included to improve the closure of pores initially, namely by introducing a system that works independently of the pH, e.g. the semihydrate of CaSO_{4,} gypsum. And a further system to solidify the total system initially, the combination of phosphoric acid and zinc oxide-forming Zn-phosphate. These phases will not contribute to the long-term properties but will enhance the initial pore closure and initial strength.

By using granules the w/c ratio (water/cement ratio) can be lower than for the loose powder. The flow-ability of the material is higher when it is granulated. The granules should preferably be of a size below 1 mm, more preferably below 0.5 mm and most preferably below 0.4 mm. The compaction density of the granule, the granule density should be above 35%, preferably above 50% most preferably above 60%.

By using such highly compacted small granules, the shaping of the material can take place in a subsequent step, without any remaining workability limitations of highly compacted bodies. A facilitated shaping in such a subsequent step, such as kneading, extrusion, tablet throwing, ultrasound etc., can be made while retaining a mobility in the system that has a high final degree of compaction, exceeding 35%, preferably exceeding 50%, even more preferred exceeding 60%.

The principle is based on the fact that a small granule—after granulation of a pre-pressed, highly compacted body—contains several tenths of millions of contact points between particles in the same, which particles are in the micrometer magnitude. When these small granules are pressed together to form new bodies, new contact points arise, which new contact points are not of the same high degree of compaction. The lower degree of compaction in these new contact points results in an improved workability, while the total degree of compaction is only marginally lowered by the lower degree of compaction in the new contact points. This is due to the new contact points only constituting a very slight proportion of the total amount of contact points. Even if for example a thousand new contact points are formed, these contact surfaces will be less than per mille of the total contact surfaces, i.e. they have a very slight influence on the end density, which will be determined by the higher degree of compaction of the granules according to the present invention. Moreover, the contact zones between individual, packed granules will hardly be distinguishable from the other contact points, as the general hardening mechanism for systems according to the invention comprises dissolution of solid material by reaction with water, which leads to the formation of ions, a saturated solution and hydrate precipitation.

In a system in which the cement hydrates due to an added liquid, the new contact points will furthermore be filled by hardened phases, which means that the homogeneity increases after the hydration/hardening. By the final degree of compaction being increased in that way, a more dense end product will be obtained, which leads to an increased strength, a possibility to lower the amount of radio-opaque agents and an easier achieved translucency, at the same time as the workability of the product is very good.

According to one aspect of this embodiment, the granules preferably exhibit a degree of compaction above 60%, even more preferred above 65% and most preferred above 70%. Preferably, the granules have a mean size of at least 30 μm, preferably at least 50 μm and even more preferred at least 70 μm, but 250 μm at the most, preferably 200 μm at the most and even more preferred 150 μm at the most, while the powder particles in the granules have a maximal particle size less than 20 μm, preferably less than 10 μm. It should hereby be noted that it is only a very slight proportion of the powder particles that constitute particles having the maximal particle size. The particle size is measured by laser diffraction. The highly compacted granules are manufactured by the powdered material being compacted to the specified degree of compaction, by cold isostatic pressing, tablet pressing of thin layers, hydro-pulse technique or explosion compacting e.g., where after the material compacted accordingly is granulated, for example crushed or tom to granules of the specified size.

The system and material according to the invention have the advantages compared to systems/materials such as glass ionomer cements and pure Ca-aluminate based systems or monomer based filling materials, that it maintains its bioactivity, that it has improved initial strength and that it has long time stability regarding both dimensional aspects, strength and minimised deterioration. The viscosity of the material can be controlled within wide ranges, upon initial mixing of the powdered material and the hydration liquid, from moist granules to an injectable slurry. The material is unique in that it solidifies in at least two steps, i.e. by cross-linking of the organic acid or salt thereof with cat-ions from both the inorganic cement system and the added reactive glass, and by hydration of one or more systems.

### EXAMPLE 1

Tests were performed to investigate the influence of amount of poly acid and the composition of the chemical bonded ceramic on the mechanical properties. The values are compared to commercial glass ionomer cement and amalgam.

### Raw Materials Used

Calcium aluminate ((CaO)₃(Al₂O₃), (CaO)(Al₂O₃), (CaO)₁₂(Al₂O₃)₇), calcium silicates (CaO)(SiO₂, (2CaO)(SiO₂), (3CaO)(SiO₂), dental glass filler (Schott), poly acid (PAA=poly acrylic acid Mw=50,000, Na-PAMA=poly(acrylic-co-maleic acid) sodium salt Mw=50,000) and reactive glasses (Schott and experimental glass). Glass ionomer cement (Fuji II, GC-corp) and Amalgam (Dispersalloy, Dentsply).

### Preparation of Material Used

Calcium aluminate was mixed with dental glass, reactive glass, poly acrylic acid and poly(acrylic-co-maleic acid) sodium salt. The calcium aluminate phases were synthesised via a sintering process, wherein first CaO and Al₂O₃ were mixed to the desired composition and then sintered at elevated temperature for 6 hours. The formed calcium aluminate lumps were crushed and jet-milled to a mean grain size of 1.5 μm and a maximum grain size of 9 μm. The dental glass, calcium aluminate and poly acids were mixed with acetone and Si₃N₄ marbles for 14 hours to obtain the desired homogeneity. The same procedure was used for the Formulation 8 using Ca silicates. Formulations were made according to (in wt. %):

| | | | | Na- | |
|---|---|---|---|---|---|
| | | Inert | Reactive | PAMA | PAA |
| Formulation | Calcium aluminate phase | glass | glass | Mw 5000 | Mw 50000 |
| 1 | (CaO)(Al₂O₃) 63.5 | 33.5 | — | 3 | — |
| 2 | (CaO)(Al₂O₃) 47 | 25 | 20 | 3 | 5 |
| 3 | (CaO)(Al₂O₃) 31 | 17 | 40 | 2 | 10 |
| 4 | (CaO)(Al₂O₃) 13 | 6 | 60 | 1 | 20 |
| 5 | (CaO)(Al₂O₃)/(CaO)₁₂(Al₂O₃)₇ | 25 | 20 | 3 | 5 |
| | mineral mixture of 90/10 and 47 | | | | |
| | in total | | | | |
| 6 | (CaO)(Al₂O₃)/(CaO)₁₂(Al₂O₃)₇ | 17 | 40 | 2 | 10 |
| | mineral mixture of 50/50 and 31 | | | | |
| | in total | | | | |
| 7 | (CaO)(Al₂O₃)/(CaO)₁₂(Al₂O₃)₇ | 5* | 42 | — | 7 |
| | mineral mixture of 50/50 and 46 | | | | |
| | in total | | | | |
| 8 | (CaO)(SiO₂)/(2CaO)(SiO₂)/ | 5 | 42 | — | 7 |
| | (3CaO)(SiO₂) mineral mixture of | | | | |
| | 45/45/10 and 46 in total | | | | |

*= inert glass as fibers

The formulations were placed in 5 ml jars and wet with liquid and blended in a “Rotomix” (3M ESPE) for 15 seconds followed by centrifugation for 3 seconds. In addition 18 mM of LiCl was added to further increase the hydration speed. The liquid contents were controlled via

$$\frac{{w}_{c}}{c} + \frac{P A A}{reactive\_glass} + \frac{{w}_{G I C}}{reactive\_glass}$$

with a w_{c}/c=0.32 (refers to the inorganic cement-system), PAA/(reactive glass)=0.14 and w/(reactive glass)=0.37 (refers to the glass ionomer system).

### Description of Tests

The diametral tensile strength was measured for the six formulations, the amalgam and the glass ionomer cement. The strength was measured after 15 min, 60 min, 4 hours and 24 hours. All samples were stored in phosphate buffer solution (pH 7.4) before measurement of DTS. The pH was measured by soaking a defined amount of material in distilled water (material/water ⅓ by volume) for the same time periods as the DTS-measurements. All storages were at 37° C.

### Results

The results of the tests were:

| | 15 min | 60 min | 4 hours | 24 hours |
|---|---|---|---|---|
| Material | (MPa)/pH | (MPa)/pH | (MPa)/pH | (MPa)/pH |
| Formulation 1 | 1.5/8 | 6.2/10 | 8.3/11 | 20.1/11.1 |
| Formulation 2 | 2.1/3.2 | 8.5/6 | 11.1/8 | 26.8/10.9 |
| Formulation 3 | 4.3/3 | 9.1/5.7 | 14.7/7.3 | 26.7/10.5 |
| Formulation 4 | 8.2/2.4 | 10.4/4.2 | 12.2/6.3 | 14/7 |
| Formulation 5 | 3.1/3 | 8.7/6.6 | 12.4/9 | 29/11.3 |
| Formulation 6 | 5.5/2.1 | 10.3/5.7 | 15.4/7.6 | 27.7/10.9 |
| Formulation 7 | 9.0/7.2 | 11.3/10.5 | 15.5/10.5 | 28.5/10.5 |
| Formulation 8 | 6.0/12.2 | 7.1/12.4 | 10.9/12.1 | 21.7/11.8 |
| Fuji II | 10.1/2 | 12.3/2.5 | 11.2/3.1 | 11.1/4 |
| Dispersalloy | 2.1/n.a. | 9.1/n.a. | 14.2/n.a. | 29.3/n.a. |

By adding PAA and reactive glass to the calcium aluminate system an increased initial strength can be achieved. Also, by adding (CaO)₁₂(Al₂O₃)₇ the reaction speed is increased and thus also the initial strength. The increase in pH over time for the formulations with calcium aluminate shows that the hydration reaction is similar to the pure calcium aluminate system.

### EXAMPLE 2

A series of tests was performed to investigate the influence of poly acid on the acid erosion resistance. The values are compared to commercial glass ionomer cement (Fuji II) and to commercial calcium aluminate based dental material (DoxaDent, Doxa AB).

### Raw Materials Used

Calcium aluminate (CaO)(Al₂O₃), dental glass filler (Schott), Na-PAMA=poly(acrylic-co-maleic acid) sodium salt, poly acrylic acid Mw 50000, reactive glass.

### Description of Tests

Test a) to c) investigated:
   a) the acid erosion of Fuji II
   b) the acid erosion of DoxaDent
   c) as formulation 3 described in Example 1.
   d) as formulation 7 described in Example 1.

The calcium aluminate phases were synthesised via a sintering process where first CaO and Al₂O₃ were mixed to the desired composition and then sintered at elevated temperature for 6 hours. The formed calcium aluminate lumps were crushed and jet-milled to a mean grain size of 3 μm and a maximum grain size of 9 μm. The dental glass, reactive glass, calcium aluminate and poly acids were mixed with acetone and Si₃N₄ marbles for 14 hours to obtain the desired homogeneity. The samples in the tests c) and d) were blended to the desired water to cement ratio in 5 ml jars and rotated at 500 rpm for 15 seconds. DoxaDent and Fuji II samples were made according to the manufactures instructions. The acid erosion was measured according to ISO-9917.

The results showed that the tests in b) and c) and d) exhibited an acid erosion of below 0.01 mm/h (below the detection limit) whereas the glass ionomer cement showed a acid erosion of 0.1 mm/h. Thus the results show that addition of poly acid to calcium aluminate does not reduce its acid resistance.

### EXAMPLE 3

A series of tests was performed to investigate the possible in vitro bioactivity of the calcium based cement material, the glass ionomer cement and the combination of the two. Bioactivity is defined herein as the ability to form apatite on the surface in contact with body fluids.

### Preparation of Materials Used

Calcium aluminate was mixed with dental glass, reactive glass, poly acrylic acid and poly(acrylic-co-maleic acid) sodium salt. The calcium aluminate phases were synthesised via a sintering process where first CaO and Al₂O₃ was mixed to the desired composition and then sintered at elevated temperature for 6 hours. The formed calcium aluminate lumps were crushed and jet-milled to a mean grain size of 2.5 μm and a maximum grain size of 9 μm. The dental glass, calcium aluminate and poly acids were mixed with acetone and Si₃N₄ marbles for 14 hours to obtain the desired homogeneity. The same procedure was used for the Formulation 8 using Ca silicates. Formulations were made according to (in wt. %):

| | | Inert | Reactive | Na-PAMA | PAA |
|---|---|---|---|---|---|
| Formulation | Calcium aluminate phase | glass | glass | Mw 5000 | Mw 50000 |
| 1 | (CaO)(Al₂O₃) 63.5 | 33.5 | — | 3 | — |
| 2 | (CaO)(Al₂O₃) 47 | 25 | 20 | 3 | 5 |
| 3 | (CaO)(Al₂O₃) 31 | 17 | 40 | 2 | 10 |
| 4 | (CaO)(Al₂O₃) 13 | 6 | 60 | 1 | 20 |
| 5 | (CaO)(Al₂O₃)/(CaO)₁₂(Al₂O₃)₇ | 25 | 20 | 3 | 5 |
| | mineral mixture of 90/10 and | | | | |
| | 47 in total | | | | |
| 6 | (CaO)(Al₂O₃)/(CaO)₁₂(Al₂O₃)₇ | 17 | 40 | 2 | 10 |
| | mineral mixture of 50/50 and | | | | |
| | 31 in total | | | | |
| 7 | (CaO)(Al₂O₃)/(CaO)₁₂(Al₂O₃)₇ | 5* | 42 | — | 7 |
| | mineral mixture of 50/50 and | | | | |
| | 46 in total | | | | |
| 8 | (CaO)(SiO)/(2CaO)(SiO₂)/ | 5 | 42 | — | 7 |
| | (3CaO)(SiO₂) mineral mixture | | | | |
| | of 45/45/10 and 46 in total | | | | |

*inert glass as glass fibers

0.5 grams of each the formulation were placed in 5 ml jars and wet with liquid and blended in a mixer by 3M/ESPE for 15 seconds followed by centrifugation for 3 seconds. In addition 18 mM of LiCl was added to further increase the hydration speed. The liquids composition were controlled via

$$\frac{{w}_{c}}{c} + \frac{P A A}{reactive\_glass} + \frac{{w}_{G I C}}{reactive\_glass}$$

with a w_{c}/c=0.32 (refers to the CBC-system), PAA/(reactive glass)=0.14 and w/(reactive glass)=0.37 (refers to the glass ionomer system). For comparison samples of GIC were also made.

### Description of Tests

The bioactivity was studied by soaking a defined amount of material in simulated body fluid (SBF) (material/SBF ⅓ by volume) for time periods of 1 day, 7 days and 21 days at 37° C. After storage the samples were removed from the SBF, rinsed in distilled water and dried at 37° C. for 48 hours. The surface composition of the formulations was studied with thin film X-ray diffraction (1° angle) and SEM combined with EDX. For each formulation and time period 5 samples were analysed. For SEM the presence of Ca and P on the surface with a ratio 1.67 indicates formation of apatite. In XRD the peaks according to the powder diffraction file for apatite must comply with the pattern from the sample.

### Results

The results from the analysis can be seen in the Table below. All formulations with calcium based cements formed apatite on the surface after 21 days. The formulations with low amounts of calcium aluminate did not form the apatite layer as quick as the formulations with much calcium aluminate, which all had apatite on the surface after 1 day. The GIC material did not form apatite on the surface. Thus the combined material can be considered bioactive.

TABLE
Results from the bioactivity tests.

| | 1 day | 7 days | 21 days |
|---|---|---|---|
| Material | XRD/SEM | XRD/SEM | XRD/SEM |
| Formulation 1 | Apatite | Apatite | Apatite |
| Formulation 2 | Apatite | Apatite | Apatite |
| Formulation 3 | Apatite | Apatite | Apatite |
| Formulation 4 | — | — | Apatite |
| Formulation 5 | Apatite | Apatite | Apatite |
| Formulation 6 | Apatite | Apatite | Apatite |
| Formulation 7 | Apatite | Apatite | Apatite |
| Formulation 8 | Apatite | Apatite | Apatite |
| Fuji II | — | — | — |

The invention is not limited to the embodiments described herein, but can be varied within the scope of the claims.

1. A system for a chemically bonded material, comprising
an aqueous hydration liquid;
a powdered material comprising a first binder phase (c), which powdered material has the capacity following saturation with the liquid reacting with said first binder phase to hydrate to a chemically bonded ceramic material;
a second, non-ceramic binder phase having a different initiation time for setting and/or a different setting rate than the initiation time for hydration and the hydration rate, respectively, of said first binder phase,
characterised in that the system further comprises a reactive glass; and
a second portion of aqueous hydration liquid (w_{GIC}), wherein
w=w_{c}+w_{GIC}
(w_{c}/c)+(second binder phase)/(reactive glass)+W_{GIC}/ (reactive glass)
with 0.2<w_{c}/c<0.45, 0<(second binder phase)/(reactive glass)<0.21 and 0.2<W_{GIC}/(reactive glass)<0.45, and in that the system provides for an ionic interaction between the hydration reactions and setting reactions of the first binder phase (c) and the second binder phase, respectively.

2. A system according to claim 1, characterised in that it is adapted to enable an initial pH to be kept <7, more preferably <4 and most preferably 1-3 to control properties related to different initiation time for setting and hardening of the part systems.

3. A system according to claim 1, characterised in that the second binder phase comprises a polycarboxylic acid and/or a copolymer or a salt or an ester thereof providing a pH value in the system of <7, preferably <4 for the first 20 minutes after mixing, preferably, a pH in the interval 1-4 for the first 10 minutes, and most preferably for the first 5 minutes.

4. A system according to claim 1, characterised in that a base is comprised in the system, so as to achieve a change of the pH to a pH>7, more preferably a pH>10, after an initial period of time after mixing of the system of a few minutes up to approximately 5 minutes at pH<7.

5. A system according to claim 1, characterised in that an additional acid is comprised in the system, so as to keep the pH<7 during a prolonged time of up to 30 minutes, preferably up to 20 minutes.

6. A system according to claim 4, characterised in that the system comprises a porous material, preferably a nano/meso-pore structure or a zeolite type structure, that is able to release said base.

7. A system according to claim 1, characterised in that particles of said first binder phase are coated with a dissolution-reducing layer, preferably comprising a glyconate.

8. A system according to claim 1, characterised in that it comprises inert filler particles composed of pre-hydrated chemically bonded ceramics, preferably of the same composition as said first binder phase.

9. A system according to claim 1, characterised in that it comprises semihydrate of CaSO₄ and/or a combination of phosphoric acid and zinc oxide-forming Zn-phosphate.

10. A system according to claim 1, characterised in that the system yields an initial strength above 5 MPa measured by diametral tensile strength after 15 minutes.

11. A powdered material for dental or orthopaedic applications for use in the system of claim 1, comprising a first binder phase essentially consisting of a cement system, which powdered material has the capacity following saturation with a hydration liquid reacting with said first binder phase to hydrate to a chemically bonded ceramic material, and an additive of a second, non-ceramic binder phase having a different initiation time for setting and/or a different setting rate than the initiation time for hydration and the hydration rate, respectively, of said first binder phase, characterised in that the powdered material comprises a reactive glass and that the second binder phase comprises a polycarboxylic acid or a copolymer or a salt or an ester thereof having a molecular weight of 100-250,000, preferably 1000-100,000, in an amount of up to 30% by weight, based on the powdered material including any dry additives.

12. A powdered material for dental applications of claim 11, characterised in that the polycarboxylic acid or a copolymer or a salt or an ester thereof, is present in an amount of 1-20%, and preferably 3-15% by weight, based on the powdered material including any dry additives.

13. A powdered material for orthopaedic applications of claim 11, characterised in that the polycarboxylic acid or a copolymer or a salt or an ester thereof is present in an amount of 1-15% and preferably 2-5% by weight, based on the powdered material including any dry additives.

14. A powdered material of claim 11, characterised in that the chemically bonded ceramic material is a material in the group that consists of aluminates, silicates, phosphates, sulphates and combinations thereof, preferably having cations in the group that consists of Ca, Sr and Ba, calcium aluminate cements being most preferred, in which case the first binder phase preferably has a composition between the phases 3CaO•Al₂O₃ and CaO•2Al₂O₃, most preferably about 12CaO•7Al₂O_{3,} optionally as glass phases.

15. A powdered material according to claim 11, characterised in that at least a part or most preferred all the reactive groups of said polycarboxylic acid or salt thereof bond to the chemically bonded ceramic material.

16. A powdered material according to claim 11, characterised in that said polycarboxylic acid or copolymer or salt or an ester thereof is a substance in the group that consists of poly acrylic acid, poly(acrylic-co-maleic acid), poly(itaconic acid), tricarballylic acid; copolymers, salts and esters thereof; and combinations thereof.

17. A powdered material according to claim 11, characterised in that it contains an inert phase additive, preferably including dental glass and preferably at a content of 3-30 weight-% more preferably 5-20%.

18. A powdered material according to claim 17, characterised in that said inert phase additive has a particle size of 0.1-5 μm, more preferably 0.2-2 μm, and most preferably 0.3-0.7 μm.

19. A powdered material according to claim 17, characterised in that said inert phase comprises low amounts of less stable phases or reactive phases including glasses, preferably below 10% of the inert phase content, which less stable phases or reactive phases preferably comprise fluoride and/or phosphorus.

20. A powdered material according to claim 11, characterised in that it has the form of granules, preferably of a size below 1 mm, more preferred below 0.5 mm and most preferred below 0.4 mm and having a granule compaction density above 35%, preferably above 50%, most preferably above 60%.

21. An aqueous hydration liquid for a powdered material comprising a first binder phase essentially consisting of a cement system, which powdered material has the capacity following saturation with the hydration liquid reacting with said first binder phase to hydrate to a chemically bonded ceramic material, characterised in that said hydration liquid comprises an additive of a second, non-ceramic binder phase, which second binder phase has a different initiation time for setting and/or a different setting rate than the initiation time for hydration and the hydration rate, respectively, of said first binder phase, and in that the hydration liquid together with the powdered material provides for an ion interaction between the hydration reactions and setting reactions of the first binder phase and the second binder phase, respectively.

22. An aqueous hydration liquid according to claim 21, characterised in that said second binder phase comprises a polycarboxylic acid or a copolymer or a salt or an ester thereof.

23. An aqueous hydration liquid according to claim 22, characterised in that at least a part or most preferred all of the reactive groups of said polycarboxylic acid or salt thereof bond to the chemically bonded ceramic material.

24. An aqueous hydration liquid according to claim 22, characterised in that said polycarboxylic acid or copolymer or salt or ester thereof is a substance in the group that consists of poly acrylic acid, poly(acrylic-co-maleic acid), poly(itaconic acid), tricarballylic acid; copolymers, salts and esters thereof; and combinations thereof.

25. An aqueous hydration liquid according to claim 22, characterised in that said polycarboxylic acid or copolymer or salt or ester thereof has a molecular weight of 100-250,000, preferably 1000-100,000.

26. An aqueous hydration liquid according to claim 21, characterised in that it has a pH of 1-7, preferably >3, before the hydration and setting reactions.

27. A chemically bonded material formed from the system of claim 1, the binder phase of which essentially consists of an inorganic cement phase and which material is in situ formed on a substrate or in a cavity, characterised in that said material also comprises a reactive, soluble glass, an in situ formed phase of polyacrylate polymer or co-polymer.

28. A system according to claim 5, characterised in that the system comprises a porous material, preferably a nano/meso-pore structure or a zeolite type structure, that is able to release said acid.
